(19) United States
(12) Patent Application Publication
(10) Pub. No.: US 2004/0235906 A1
(43) Pub. Date: Nov. 25, 2004

(54) Substituted amine derivatives and their use as monoamine neurotransmitter re-up-take inhibitors

(76) Inventors: Alex Haahr Gouliaev, Vekso Sj (DK); Dan Peters, Ballerup (DK)

Correspondence Address: BIRCH STEWART KOLASCH & BIRCH, PO BOX 747, FALLS CHURCH, VA, 22040-0747, US

(21) Appl. No.: 10/485,314

(22) PCT Filed: Aug. 1, 2002
(86) PCT No.: PCT/DK02/00518

(30) Foreign Application Priority Data
Aug. 8, 2001 (DK) PA 2001 01199

(51) Int. Cl. C07D 213/78; A61K 31/44; A61K 31/4025

(52) U.S. Cl. 514/347; 514/424; 514/651; 546/300; 548/550; 564/347

(57) ABSTRACT

This invention relates to novel substituted amine derivatives. In other aspects the invention relates to the use of these compounds in a method for therapy and to pharmaceutical compositions comprising the compounds of the invention.

### TECHNICAL FIELD

This invention relates to novel substituted amine derivatives.

In other aspects the invention relates to the use of these compounds in a method for therapy and to pharmaceutical compositions comprising the compounds of the invention.

### BACKGROUND ART

Prior art describes a number of compounds active as mixed monoamine neurotransmitter re-uptake inhibitors. Examples are WO 97/30997 describing tropane derivatives and WO 97/16451 describing fused tropane-derivatives.

However, there is a continued strong need to find compounds with an optimised biochemical profile as regards the activity on reuptake of the monoamine neurotransmitters serotonin, dopamine and noradrenaline, such as the ratio of the serotonin reuptake versus the noradrenaline and dopamine activity.

### SUMMARY OF THE INVENTION

Therefore, in its first aspect, the invention provides a compound of general formula I,

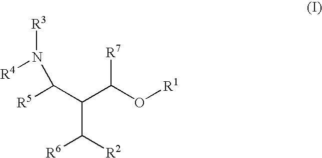

or a pharmaceutically acceptable addition salt thereof or the N-oxide thereof.

In its second aspect the invention provides a pharmaceutical composition comprising a therapeutically effective amount of a compound of the invention together with at least one pharmaceutically-acceptable carrier, excipient or diluent.

In a further aspect the invention provides a method of treatment, prevention or alleviation of a disease or a disorder or a condition of a living animal body, including a human, which disorder, disease or condition is responsive to inhibition of monoamine neurotransmitter re-uptake in the central nervous system (CNS), which method comprises the step of administering to such a living animal body in need thereof a therapeutically effective amount of a compound of the invention.

Other objects of the invention will be apparent to the person skilled in the art from the following detailed description and examples.

### DETAILED DISCLOSURE OF THE INVENTION

In its first aspect, the invention provides a compound of general formula I,

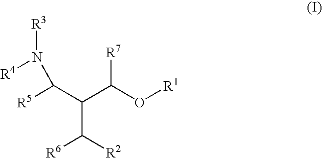

or a pharmaceutically acceptable addition salt thereof or the N-oxide thereof wherein

R¹ is aryl or heteroaryl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R² is hydrogen, alkyl, cycloalkyl, alkenyl, alkynyl, —O-alkyl, —S-alkyl, —NR⁸-alkyl, aryl or heteroaryl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl; and

with the proviso that when R² is hydrogen, optionally substituted aryl or optionally substituted heteroaryl, then R¹ is 3,4-dichlorophenyl;

R⁸ is hydrogen, alkyl, alkenyl, or alkynyl; and

R³ is hydrogen, alkyl, alkenyl, alkynyl, cycloalkyl, cycloalkylalkyl, 2-hydroxyethyl, aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R⁴ is hydrogen, alkyl, alkenyl, alkynyl, cycloalkyl, cycloalkylalkyl, 2-hydroxyethyl, aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R⁵ is hydrogen;

R⁶ is hydrogen;

R⁷ is hydrogen; or

one of R³ and R⁴ form together with R⁶—(CH₂)ₚ—;

wherein p is 2 or 3;

the other of R³ and R⁴ is hydrogen, alkyl, alkenyl, alkynyl, cycloalkyl, cycloalkylalkyl, 2-hydroxyethyl, aryl, arylalkyl, heteroaryl or heteroarylalkyl; wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R⁵ is hydrogen;

R⁷ is hydrogen; or

one of R³ and R⁴ form together with R⁶—(CH₂)_{q}—CR⁹—(CH₂)ᵣ—;

wherein q is 0 or 1;

r is 0 or 1;

the other of R³ and R⁴ is hydrogen, alkyl, alkenyl, alkynyl, cycloalkyl, cycloalkylalkyl, 2-hydroxyethyl, aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R⁹ together with R⁵ form —(CH₂)ₛ—;

wherein s is 2 or 3;

R⁷ is hydrogen; or

one of R³ and R⁴ form together with R⁶—(CH₂)_{q}—CR⁹—(CH₂)ᵣ—;

wherein q is 0 or 1;

r is 0 or 1;

R⁹ together with R⁵ form —(CH₂)ₛ—;

wherein s is 1, 2, or 3;

the other of R³ and R⁴ form together with R⁷—(CH₂)ₜ—;

wherein t is 1, 2, 3, or 4.

In its second aspect, the invention provides a pharmaceutical composition, comprising a therapeutically effective amount of a compound of the invention, or a pharmaceutically acceptable addition salt thereof, together with at least one pharmaceutically acceptable carrier, excipient or diluent.

In a further aspect, the invention provides the use of a compound of the invention, or a pharmaceutically acceptable addition salt thereof, for the manufacture of a pharmaceutical composition for the treatment, prevention or alleviation of a disease or a disorder or a condition of a mammal, including a human, which disease, disorder or condition is responsive to inhibition of monoamine neurotransmifter re-uptake in the central nervous system.

In a still further aspect, the invention relates to a method for treatment, prevention or alleviation of a disease or a disorder or a condition of a living animal body, including a human, which disorder, disease or condition is responsive to inhibition of monoamine neurotransmitter re-uptake in the central nervous system, which method comprises the step of administering to such a living animal body in need thereof a therapeutically effective amount of a compound of the invention.

In one embodiment of the compound of general formula I, R¹ is optionally substituted aryl, such as optionally substituted phenyl. In a special embodiment, R¹ is di-chlorosubstituted phenyl, such as 3,4-dichlorophenyl. In a further embodiment, R¹ is optionally substituted heteroaryl, such as optionally substituted pyridazinyl, pyrazinyl, benzothiazolyl, or quinolinyl. In a special embodiment, R¹ is optionally substituted pyridazinyl, such as pyridazinyl or chloropyridazinyl, in particular pyridazin-3-yl or 6-chloropyridazin-3-yl. In a further special embodiment, R¹ is optionally substituted pyrazinyl, such as pyrazinyl or chloropyrazinyl, in particular pyrazin-2-yl or 6-chloropyrazin-2-yl. In a still further special embodiment, R¹ is optionally substituted benzothiazolyl, such as benzothiazolyl or chlorobenzothiazolyl, in particular benzothiazol-2-yl or 6-chlorobenzothiazol-2-yl. In a further special embodiment, R¹ is optionally substituted quinolinyl, such as quinolinyl or nitroquinolinyl, in particular quinolin-2-yl or 6-nitro-quinolin-2-yl.

In a further embodiment, R² is hydrogen, alkyl, alkenyl, alkynyl, —O-alkyl, —S-alkyl, —NR⁸-alkyl, aryl or heteroaryl; wherein the aryl and heteroaryl are optionally substituted with one or more substituents selected from the group consisting of: halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl.

In a still further embodiment of the compound of general formula I, R² is alkyl, alkenyl, alkynyl, —O-alkyl, —S-alkyl, or —NR⁸-alkyl. In a special embodiment, R² is methyl, ethyl, propyl, isopropyl, butyl, isobutyl, or t-butyl. In a further embodiment, R² is methyl, In a still further embodiment R² is ethyl, In a further embodiment, R² is propyl. In a still further embodiment, R² is isopropyl. In a further embodiment, R² is butyl. In a still further special embodiment of the compound of general formula I, R² is —O-alkyl. In a special embodiment, R² is —O-isopropyl. In a further special embodiment of the compound of general formula I, R² is —S-alkyl. In a special embodiment, R² is —S-isopropyl. In a further special embodiment of the compound of general formula I, R² is —NR⁸-alkyl, such as —NH-alkyl. In a special embodiment, R is —NH-isopropyl.

In a further embodiment of the compound of general formula I, R² is hydrogen .

In a still further embodiment of the compound of general formula I, R² is optionally substituted aryl or optionally substituted heteroaryl. In a special embodiment, R² is optionally substituted aryl, such as optionally substituted phenyl. In a further special embodiment, R² is di-chlorosubstituted phenyl, such as 3,4-dichlorophenyl. In a still further special embodiment, R² is phenyl.

In a further embodiment, R² is cycloalkyl, such as cycloheptyl.

In a still further embodiment of the compound of general formula I, R² is hydrogen and R¹ is 3,4-dichlorophenyl. In a further embodiment of the compound of general formula I, R² is optionally substituted aryl and R¹ is 3,4-dichlorophenyl. In a still further embodiment of the compound of general formula I, R² is optionally substituted heteroaryl and R¹ is 3,4-dichlorophenyl.

In a further embodiment of the compound of general formula I, R² is alkyl, alkenyl, alkynyl, —O-alkyl, —S-alkyl, —NR⁸-alkyl, aryl or heteroaryl. In a still further embodiment of the compound of general formula I, R² is hydrogen, alkyl, alkenyl, alkynyl, —O-alkyl, —S-alkyl, —NR⁸-alkyl, or heteroaryl. In a further embodiment of the compound of general formula I, R² is hydrogen, alkyl, alkenyl, alkynyl, —O-alkyl, —S-alkyl, —NR⁸-alkyl, or aryl.

In a still further embodiment of the compound of general formula I, R⁸ is hydrogen or alkyl. In one embodiment, R⁸ is hydrogen. In a further embodiment, R⁸ is alkyl.

In a further embodiment of the compound of general formula I, R³ is hydrogen, alkyl, alkenyl, alkynyl, cycloalkyl, cycloalkylalkyl, 2-hydroxyethyl, aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl.

In a still further embodiment of the compound of general formula I, R³ form together with R⁶—(CH₂)ₚ—; wherein p is 2 or 3. In a special embodiment, R³ form together with R⁶—(CH₂)₂—. In a further special embodiment, R³ form together with R⁶—(CH₂)₃—.

In a further embodiment of the compound of general formula I, R³ form together with R⁶—(CH₂)_{q}—CR⁹—(CH₂)ᵣ—; wherein q is 0 or 1; and r is 0 or 1. In a special embodiment, R³ form together with R⁶—CR⁹—(CH₂)—.

In a still further embodiment of the compound of general formula I, R⁴ is hydrogen, alkyl, alkenyl, alkynyl, cycloalkyl, cycloalkylalkyl, 2-hydroxyethyl, aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl.

In a further embodiment of the compound of general formula I, R⁴ form together with R⁶—(CH₂)ₚ—; wherein p is 2 or 3. In a special embodiment, R⁴ form together with R⁶—(CH₂)₂—. In a further special embodiment, R⁴ form together with R⁶—(CH₂)₃—.

In a still further embodiment of the compound of general formula I, R⁴ form together with R⁶—(CH₂)_{q}—CR⁹—(CH₂)ᵣ—; wherein q is 0 or 1; and r is 0 or 1. In a special embodiment, R⁴ form together with R⁶—CR⁹—(CH₂)—.

In a further embodiment of the compound of general formula I, the other of R³ and R⁴ is hydrogen or alkyl. In a special embodiment, the other of R³ and R⁴ is hydrogen. In a further special embodiment, the other of R³ and R⁴ is methyl. In a still further special embodiment, the other of R³ and R⁴ is ethyl. In a further special embodiment, the other of R³ and R⁴ is propyl, such as isopropyl.

In a further embodiment of the compound of general formula I, R⁵ is hydrogen.

In a still further embodiment of the compound of general formula I, R⁵ together with R⁹ form —(CH₂)ₛ—; wherein s is 2 or 3. In a special embodiment, R⁵ together with R⁹ form —(CH₂)₂—. In a further special embodiment, R⁵ together with R⁹ form —(CH₂)₃—.

In a further embodiment of the compound of general formula I, R⁶ is hydrogen.

In a still further embodiment of the compound of general formula I, R⁷ is hydrogen.

In a further embodiment of the compound of general formula I, R³ form together with R⁷—(CH₂)ₜ—; wherein t is 1, 2, 3, or 4. In a special embodiment, R³ form together with R⁷—(CH₂)— or —(CH₂)₂—.

In a still further embodiment of the compound of general formula I, R⁴ form together with R⁷—(CH₂)ₜ—; wherein t is 1, 2, 3, or 4. In a special embodiment, R⁴ form together with R⁷—(CH₂)— or —(CH₂)₂—.

In a further embodiment of the compound of formula I,

R³ is hydrogen, alkyl, alkenyl, alkynyl, cycloalkyl, cycloalkylalkyl, 2-hydroxyethyl, aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R⁴ is hydrogen, alkyl, alkenyl, alkynyl, cycloalkyl, cycloalkylalkyl, 2-hydroxyethyl aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R⁵ is hydrogen;

R⁶ is hydrogen;

R⁷ is hydrogen;

and R¹, R², and R⁸ are defined as above.

In a still further embodiment of the compound of formula I,

one of R³ and R⁴ form together with R⁶—(CH₂)ₚ—;

wherein p is 2 or 3;

the other of R³ and R⁴ is hydrogen, alkyl, alkenyl, alkynyl, cycloalkyl, cycloalkylalkyl, 2-hydroxyethyl, aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R⁵ is hydrogen;

R⁷ is hydrogen;

and R¹, R², and R⁸ are defined as above.

In a special embodiment of the compound of formula I, the compound is of general formula (II)

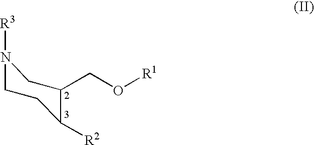

wherein R¹, R², and R³ are defined as above.

In one embodiment of the compound of general formula II, R² is hydrogen and R¹ is 3,4-dichlorophenyl. In a further embodiment of the compound of general formula II, R² is optionally substituted aryl and R¹ is 3,4-dichlorophenyl. In a still further embodiment of the compound of general formula II, R² is optionally substituted heteroaryl and R¹ is 3,4-dichlorophenyl.

It will be appreciated by those skilled in the art that the compounds of the present invention may contain one or more chiral centres and that such compounds exist in the form of isomers, i.e. 1R/S and 2R/S. Moreover, the substituent R¹—O—CH₂— on position 2 and the substituent R² on position 3 of the tropane skeleton of formula II may in particular be in cis or trans configuration relative to each another. In a preferred embodiment of the invention the substituents at positions 2 and 3 are in trans configuration. In another preferred embodiment of the invention the substituents at positions 2 and 3 are in cis configuration. The invention includes all such isomers and any mixtures thereof including racemic mixtures.

In a further embodiment of the compound of general formula I,

one of R³ and R⁴form together with R⁶—(CH₂)_{q}—CR⁹—(CH₂)ᵣ—;

wherein q is 0 or 1;

r is 0 or 1;

the other of R³ and R⁴ is hydrogen, alkyl, alkenyl, alkynyl, cycloalkyl, cycloalkylalkyl, 2-hydroxyethyl, aryl, arylalkyl, heteroaryl or heteroarylalkyl;

wherein the aryl and heteroaryl are optionally substituted with one or more substituents selected from the group consisting of:

halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;

R⁹ together with R⁵ form —(CH₂)ₛ—;

wherein s is 2 or 3;

R⁷ is hydrogen;

and R¹, R², and R⁸ are defined as above.

In a special embodiment of the compound of formula I, the compound is of general formula (III)

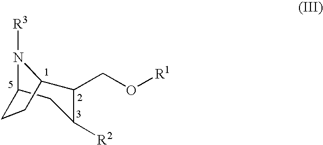

wherein R¹, R², and R³ are defined as above.

In one embodiment of the compound of general formula III,

R¹ is aryl or heteroaryl; wherein the aryl and heteroaryl are optionally substituted with one or more substituents selected from the group consisting of:

halogen, hydroxy, amino, cyano, nitro, trifluoromethyl and alkyl;

R² is hydrogen, alkyl, cycloalkyl, or aryl; wherein the aryl is optionally substituted with one or more substituents selected from the group consisting of:

halogen, hydroxy, amino, cyano, nitro, trifluoromethyl, and alkyl;

with the proviso that when R² is hydrogen or optionally substituted aryl, then R¹ is 3,4-dichlorophenyl; and

R³ is hydrogen or alkyl.

In a special embodiment of the compound of general formula III,

R¹ is aryl or heteroaryl; wherein the aryl and heteroaryl are optionally substituted with one or more substituents selected from the group consisting of halogen or nitro;

R² is hydrogen, alkyl, cycloalkyl, or aryl; wherein the aryl is optionally substituted with one or more halogen;

with the proviso that when R² is hydrogen or optionally substituted aryl, then R¹ is 3,4-dichlorophenyl; and

R³ is hydrogen or alkyl.

In a further embodiment of the compound of general formula III, R² is hydrogen and R¹ is 3,4-dichlorophenyl. In a further embodiment of the compound of general formula III, R² is optionally substituted aryl and R¹ is 3,4-dichlorophenyl. In a special embodiment R² is phenyl, In a further special embodiment, R² is 3,4-dichlorophenyl. In a still further embodiment of the compound of general formula III, R² is optionally substituted heteroaryl and R¹ is 3,4-dichlorophenyl. In a further embodiment of the compound of general formula III, R² is alkyl, such as propyl or butyl, and R¹ is 3,4-dichlorophenyl. In a still further embodiment of the compound of general formula III, R² is cycloalkyl, such as cycloheptyl, and R¹ is 3,4-dichlorophenyl. In a further embodiment of the compound of general formula III, R² is alkyl, such as propyl, and R¹ is optionally substituted heteroaryl, such as optionally substituted pyridazinyl, pyrazinyl, benzothiazolyl, or quinolinyl. In a special embodiment thereof, R¹ is pyridazinyl, such as pyridazin-3-yl, chloropyridazinyl, such as 6-chloropyridazin-3-yl, pyrazinyl, such as pyrazin-2-yl, chloropyrazinyl, such as 6-chloropyrazin-2-yl, benzothiazolyl, such as benzothiazol-2-yl, chlorobenzothiazolyl, such as 6-chlorobenzothiazol-2-yl, quinolinyl, such as quinolin-2-yl, or nitroquinolinyl, such as 6-nitro-quinolin-2-yl.

It will be appreciated by those skilled in the art that the compounds of the present invention may contain one or more chiral centers, and that such compounds exist in the form of isomers, i.e. 1R/S, 2R/S, 3R/S and 5R/S. Moreover, the substituent R¹—O—CH₂— on position 2 and the substituent R² on position 3 of the tropane skeleton of formula III may in particular be in cis or trans configuration relative to each another. In a preferred embodiment of the invention the substituents at positions 2 and 3 are in trans configuration. In another preferred embodiment of the invention the substituents at positions 2 and 3 are in cis configuration. The invention includes all such isomers and any mixtures thereof including racemic mixtures.

In a further embodiment of the compound of formula I,

one of R³ and R⁴ form together with R⁶—(CH₂)_{q}—CR⁹—(CH₂)ᵣ—;

wherein q is 0 or 1;

r is 0 or 1;

R⁹ together with R⁵ form —(CH₂)ₛ—;

wherein s is 1, 2, or 3;

the other of R³ and R⁴ form together with R⁷—(CH₂)ₜ—;

wherein t is 1, 2, 3, or 4;

and R¹, R², and R⁸ are defined as above.

In a special embodiment of the compound of formula I, the compound is of general formula (IV)

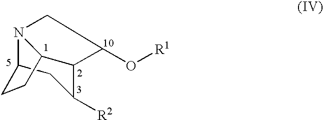

wherein R¹ and R² are defined as above.

It will be appreciated by those skilled in the art that the compounds of the present invention may contain one or more chiral centers, and that such compounds exist in the form of isomers, i.e 1R/S, 2R/S, 3R/S, 5R/S and 10R/S. The invention includes all such isomers and any mixtures thereof including racemic mixtures.

In a special embodiment, the compound of general formula I is selected from:

(2R,3S)-2-(3,4-Dichlorophenoxymethyl)-8-methyl-3-phenyl-8-aza-bicyclo[3.2.1]octane;

(2S,3R)-2-(3,4-Dichloro-phenoxymethyl)-8-methyl-3-phenyl-8-aza-bicyclo[3.2.1]octane;

(2R)-2-(3,4-Dichloro-phenoxymethyl)-8-methyl-8-aza-bicyclo[3.2.1]octane;

(1S,2S, 5R)-2-(3,4-Dichloro-phenoxymethyl)-8-methyl-8-aza-bicyclo[3.2.1]octane;

(2R,3S)-2-(3,4-Dichloro-phenoxymethyl)-3-(3,4-dichloro-phenyl)-8-methyl-8-aza-bicyclo[3.2.1]octane;

(2S,3R)-2-(3,4-Dichloro-phenoxymethyl)-3-(3,4-dichloro-phenyl8-methyl-8-aza-bicyclo[3.2.1]octane;

(2R,3S)-2-(3,4-Dichloro-phenoxymethyl)-3-(2-propyl)-8-methyl-8-aza-bicyclo[3.2.1]octane;

(1S,2S, 3R, 5R)-2-(3,4-Dichloro-phenoxymethyl)-3-(2-propyl)-8-methyl-8-aza-bicyclo[3.2.1]octane;

(2R,3S)-2-(3,4-Dichloro-phenoxymethyl)-3-(2-propyl)-8-H-8-aza-bicyclo[3.2.1]octane;

(2R,3S)-2-(3,4-Dichloro-phenoxymethyl)-3-(2-propyl)-8-(2-propyl)-8-aza-bicyclo[3.2.1]octane;

(2R,3S)-2-(3,4-Dichloro-phenoxymethyl3-(1-butyl)-8-(2-propyl)-8-aza-bicyclo[3.2.1]octane;

(2R,3S)-2-(3,4-Dichloro-phenoxymethyl)-3-cycloheptyl-8-(2-propyl)-8-aza-bicyclo[3.2.1]octane;

(2R,3S)-2-(3,4-Dichloro-phenoxymethyl)-3-(cycloheptyl)-8-methyl-8-aza-bicyclo[3.2.1]octane;

(2R,3S)-2-(3,4-Dichloro-phenoxymethyl)-3-cycloheptyl-8-H-8-aza-bicyclo[3.2.1]octane;

(2R,3S)-2-(3,4-Dichloro-phenoxymethyl)-3-(1-butyl)-8-methyl-8-aza-bicyclo[3.2.1]octane;

(2R,3S)-2-(3,4-Dichloro-phenoxymethyl)-3-(1-butyl)-8-H-8-aza-bicyclo[3.2.1]octane;

(2R,3S)-2-(6-Chloropyridazin-3-yloxymethyl)-3-(2-propyl)-8-methyl-8-aza-bicyclo[3.2.1]octane;

(2R,3S)-2-(Pyrazin-2-yloxymethyl)-3-(2-propyl)-8-methyl-8-aza-bicyclo[3.2.1]octane;

(2R,3S)-2-(6-Chlorobenzothiazol-2-yloxymethyl)-3-(2-propyl)-8-methyl-8-aza-bicyclo[3.2.1]octane;

(2R,3S)-2-(6-Nitroquinolin-2-yloxymethyl)-3-(2-propyl)-8-methyl-8-aza-bicyclo[3.2.1]octane;

(2R,3S)-2-(6-Chlorobenzothiazol-2-yloxymethyl)-3-(2-propyl)-8-H-8-aza-bicyclo[3.2.1]octane;

(2R,3S)-2-(6-Chloro-pyrazin-2-yloxymethyl)-3-(2-propyl)-8-methyl-8-aza-bicyclo[3.2.1]octane;

or a pharmaceutically acceptable addition salt thereof.

Definition of Substituents

In the context of this invention halogen represents a fluorine, a chlorine, a bromine or an iodine atom.

Alkyl means a straight chain or branched chain of one to six carbon atoms, including but not limited to, methyl, ethyl, propyl, isopropyl, butyl, isobutyl, t-butyl, pentyl, and hexyl; methyl, ethyl, propyl and isopropyl are preferred groups.

Cycloalkyl means cyclic alkyl of three to seven carbon atoms, including but not limited to cyclopropyl, cyclobutyl, cyclopentyl, and cyclohexyl;

Alkenyl means a group of from two to six carbon atoms, including at least one double bond, for example, but not limited to ethenyl, 1,2- or 2,3-propenyl, or 1,2-, 2,3-, or 3,4-butenyl.

Alkynyl means a group of from two to six carbon atoms, including at least one triple bond, for example, but not limited to ethynyl, 1,2-, 2,3-propynyl, or 1,2-, 2,3- or 3,4-butynyl.

Alkoxy is O-alkyl, wherein alkyl is as defined above.

Cycloalkoxy means O-cycloalkyl, wherein cycloalkyl is as defined above.

Cycloalkylalkyl means cycloalkyl as above and alkyl as above, meaning for example, cyclopropylmethyl.

Amino is NH₂ or NH-alkyl or N-(alkyl)₂, wherein alkyl is as defined above.

Aryl is a carbocyclic aromatic ring system such as phenyl or naphthyl (1-naphthyl or 2-naphthyl).

Heteroaryl is an aromatic mono-, bi- or poly-heterocyclic group, which holds one or more heteroatoms in its ring structure. Preferred heteroatoms include nitrogen (N), oxygen (O), and sulphur (S).

Preferred monocyclic heteroaryl groups of the invention include aromatic 5- and 6 membered heterocyclic monocyclic groups, including for example, but not limited to, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-oxadiazol-3-yl, 1,2,4-oxadiazol-5-yl, 1,2,4-thiadiazol-3-yl, 1,2,4-thiadiazol-5-yl, 1,2,5-oxadiazol-3-yl, 1,2,5-oxadiazol-4-yl, 1,2,5-thiadiazol-3-yl, 1,2,5-thiadiazol4-yl, 2-imidazolyl, 4-imidazolyl, 5-imidazolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyrimidyl, 4-pyrimidyl, 5-pyrimidyl or 6-pyrimidyl.

Preferred bicyclic heteroaryl groups of the invention include indolizinyl, in particular 2-, 5- or 6-indolizinyl; indolyl, in particular 2-, 5- or 6-indolyl; isoindolyl, in particular 2-, 5- or 6-isoindolyl; benzo[b]furanyl, in particular 2-, 5- or 6-benzofuranyl; benzo[b]thienyl, in particular 2-, 5- or 6-benzothienyl; benzimidazolyl, in particular 2-, 5- or 6-benzimidazolyl; benzothiazolyl, in particular 5- or 6-benzothiazolyl; purinyl, in particular 2- or 8-purinyl; quinolinyl, in particular 2-, 3-, 6- or 7-quinolinyl; isoquinolinyl, in particular 3-, 6- or 7-isoquinolinyl; cinnolinyl, in particular 6- or 7-cinnolinyl; phthalazinyl, in particular 6- or 7-phthalazinyl; quinazolinyl, in particular 2-, 6- or 7-quinazolinyl; quinoxalinyl, in particular 2- or 6-quinoxalinyl; 1,8-naphthyridinyl, in particular 1,8-naphthyridin-2-, 3-, 6- or 7-yl; pteridinyl, in particular 2-, 6- or 7-pteridinyl; and indenyl, in particular 1-, 2-, 3-, 5- or 5-indenyl.

Methods for the resolvation of optical isomers, known to those skilled in the art may be used, and will be apparent to the average worker skilled in the art. Such methods include those discussed by J. Jaques, A. Collet, and S. Wilen in “Enantiomers, Racemates, and Resolutions”, John Wiley and Sons, New York (1981).

The compounds of the invention may be prepared in numerous ways. The compounds of the invention and their pharmaceutically acceptable derivatives may thus be prepared by any method known in the art for the preparation of compounds of analogous structure, and as shown in the representative examples which follow.

Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention includes alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

The term “prodrug” denotes a compound, which is a drug precursor and which, following administration and absorption, release the drug in vivo via some metabolic process.

Particularly favoured prodrugs are those that increase the bioavailability of the compounds of the invention (e.g. by allowing an orally administrered compound to be more readily absorbed into the blood) or which enhance delivery of the parent compound to a specific biological compartment (e.g. the brain or lymphatic system).

Thus examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvents such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Labelled Compounds

The compounds of the invention may be used in their labelled or unlabelled form. In the context of this invention “label” stands for the binding of a marker to the compound of interest that will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

Methods of Preparation

The compounds of the invention may be prepared by conventional methods for chemical synthesis, e.g. as described below in the general synthetic route or in the working examples. The starting materials for the processes described in the present application are known or may readily be prepared by conventional methods from commercially available chemicals.

The scheme shown below, which is not intended to be in any way limiting to the scope of the invention as claimed, illustrates a general synthetic route to the compounds described in this invention.

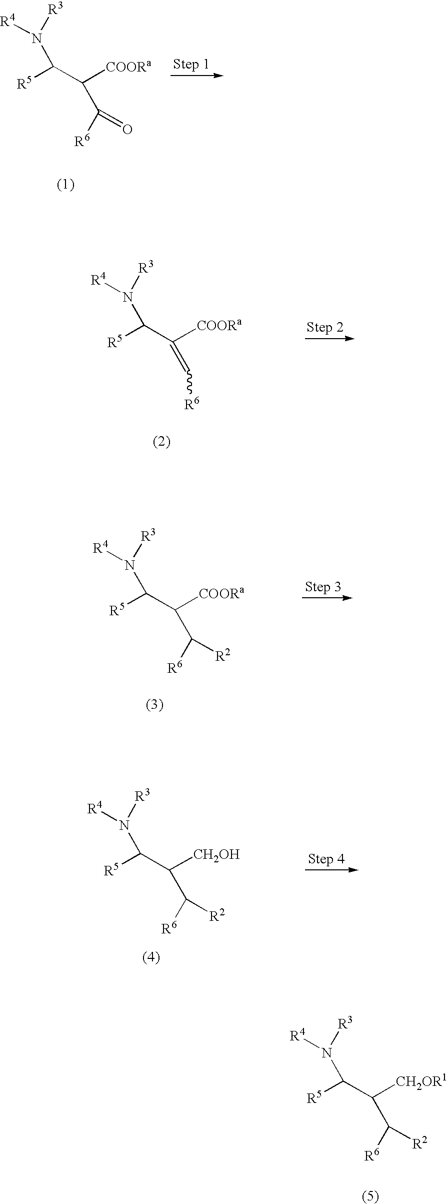

The synthetic route may lead to enantiomerically pure product, which is available by e.g. resolution of a racemate, via the use of a chiral salt such as e.g. D- or L-tartaric acid. Reduction of the ketone or aldehyde in a β-keto ester (1) by e.g. NaBH₄ followed by elimination of H₂O, catalysed by e.g. acid or base, leads to formation of an α,β-unsaturated ester (2).

The α,β-unsaturated ester (2) is then subjected to a nucleophilic 1,4-conjungate addition, whereby different R²-groups may be introduced with or without catalysis. The use of e.g. alkyl lithiums or alkyl magnesium halides leads to products, wherein R² is an alkyl group, the use of nucleophilic hydride reagents such as e.g. NaBH₄ leads to products, wherein R² is a hydrogen, the use of alcohols, thiols or amines leads to products, wherein R² is an alkoxy, a thioalkoxy or an alkylamino group. The stereoisomerism in product (3) may be controlled, by the exact choice of reaction and quench conditions. The quench of the 1,4-conjungate addition of a Grignard reagent like i-PrMgCl, using mildly acidic conditions, leads e.g. mainly to the cis-isomer, whereas stronger acidic conditions mainly leads to the trans-isomer. Furthermore, the cis-isomer may be isomerised to the trans-isomer by base catalysis.

The ester group in (3) is reduced by use of e.g. Red-Al or LiAlH₄ yielding the alcohol (4), which is transformed to the alkyl bromide, the alkyl iodide or the alkyl tosylate. Reaction of this intermediate with phenol, substituted phenols or hydroxyheteroaromatic compounds leads to the product (5) (R¹ being aryl, substituted aryl or heteroaryl). The product (5) may easily be further transformed into an appropriate salt. The structure of the N-substituent R³ or R⁴ may be controlled either from the starting material or by demethylation of an N-methyl compound using e.g. chloroethylchloroformate, followed by N-alkylation using alkylhalides, arylalkylhalides alkyltosylates, arylalkyltosylates, alkyltriflates or arylalkyltriflates, or altematively N-arylated by Pd-catalysed coupling using the corresponding aryl- or heterarylhalide.

The end product of the reaction described herein may be isolated by conventional techniques, e.g. by extraction, crystallisation, distillation, chromatography, etc.

Biological Activity

The compounds of the invention may be tested for its ability to inhibit the reuptake of the monoamine neurotransmitters in synaptosomes, eg such as described in WO 97/16451. Based on the balanced activity observed in these tests the compound of the invention is considered useful for combating diseases, disorders or conditions associated with the dopaminergic, noradrenalinergic and/or serotonergic neural system.

The diseases, disorders or conditions contemplated in this context are eating disorders, obesity, anorexia nervosa, disorders of sleep, panic disorders, social phobia, dementia, senile dementia, pre-senile dementia, memory deficits, memory loss, Alzheimers disease, chronic fatigue syndrome, anxiety, pseudodementia, Ganser's syndrome, narcolepsy, drug addiction or misuse, alcoholism, tobacco abuse, panic disorder, post-traumatic syndrome, migraine, pain, attention deficit hyperactivity disorder, autism, mutism, trichotillomania, Parkinson's disease, depression, attention, alertness, arousal, vigilance, premature ejaculation, and erectile dysfunction.

The compounds of the invention are considered particularly useful for the treatment, prevention or alleviation of depression, pseudodementia, Ganser's syndrome, obsessive compulsive disorders, panic disorders, memory deficits, attention deficit hyperactivity disorder, obesity, anxiety and eating disorders.

Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers therefore, and, optionally, other therapeutic and/or prophylactic ingredients. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.01 to about 500 mg of active ingredient per individual dose, preferably of from about 0.1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.01 μg/kg i.v. and 0.1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Methods of Therapy

In another aspect the invention provides a method for the treatment, prevention or alleviation of a disease or a disorder or a condition of a living animal body, including a human, which disease, disorder or condition is responsive to inhibition of monoamine neurotransmitter re-uptake in the central nervous system (CNS), and which method comprises administering to such a living animal body, including a human, in need thereof an effective amount of the compound of the invention.

In a more preferred embodiment the invention provides a method of combating depression, pseudodementia, Ganser's syndrome, obsessive compulsive disorders, panic disorders, memory deficits, attention deficit hyperactivity disorder, obesity, anxiety and eating disorders.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge. A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples which are not intended to be in any way limiting to the scope of the invention as claimed.

### Example 1

Formation of Starting Material

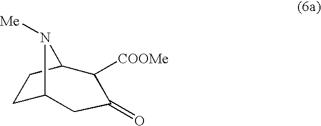

(+)-2-Carbomethoxytropinone (6a): Was prepared by a known procedure (J. F. Casale, Forensic Science International, 33 (1987) 275-298).

(−)-2-Carbomethoxytropinone (6b): Was synthesised similarly.

### Example 2

General Procedure for Conducting Step 1

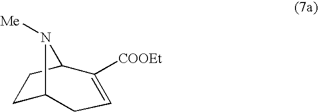

(−)-Ecgonine ethylester (7a): To a stirred solution of (+)-2-carbomethoxytropinone (6a) (37.4 g) in methyl alcohol (1.5 L) at −45° C., was added sodium borohydride (37 g) in small portions, such that the internal temperature was kept between −45° C. and −35° C. The reaction mixture was stirred at −45° C. for 2 hours, and quenched by drop wise addition of hydrochloric acid (120 mL), while keeping the temperature at −45° C. The reaction mixture was allowed to warm to room temperature and stirred overnight. The reaction mixture was concentrated to a volume of approximately 120 mL, added water (500 mL) and washed with diethyl ether (3×100 mL). The aqueous phase was added 25% ammonia (aq.) until pH 10-11, and extracted with dichloromethane (4×200 mL).

The combined organic phases were dried (sodium sulfate) and evaporated to an oil.

The oil was dissolved in ethyl acetate (370 mL) and a solution of sodium ethoxide (from 7 g of sodium) was added. The resulting solution was heated at reflux for 3 hours, cooled to r.t. and evaporated to an oil. The residue was added toluene (0.5 L) and evaporated to an oil, this was repeated. The product 30 g (79%) is an oil.

(+)-Ecgonine ethylester (7b): Was synthesised similarly from 6b.

### Example 3

General Procedure for Conducting Step 2 Using Hydride as Nucleophile

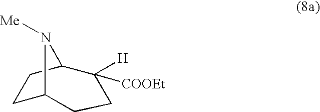

(2R)-8-Methyl-8-aza-bicyclo[3.2.1]octane-2-carboxylic acid ethyl ester (8a): A suspension of 7a (1.0 g; 5.1 mmol) and 5% Pd/C (100 mg) in THF (20 ml) was hydrogenated at 1 atm. for 1.5 h. The reaction mixture was filtered through celite and evaporated to dryness to afford 8a (1.1 g) as a clear oil. GCMS>95% purity m/z 197.

(2S)-8-Methyl-8-aza-bicyclo[3.2.1]octane-2-carboxylic acid ethyl ester (8b): Was synthesised similarly from 7b.

### Example 4

General Procedure for Conducting Step 2 Using a Grignard Reagent as Nucleophile

This procedure may lead to formation of isomers, e.g. with the alkoxycarbonyl substituent in the exo- or endoposition. These may be separated by column chromatography. When using alkylmagnesium halides, formation of isomers with respect to the 3-position is also present. These may be separated by column chromatography.

Commercially solutions of Grignard reagents were used when available, otherwise the Grignard reagent was synthesised by standard means by addition of an arylhalide or alkylhalide such as e.g. phenylbromide to a suspension of Mg in anhydrous Et₂O and used immediately after formation.

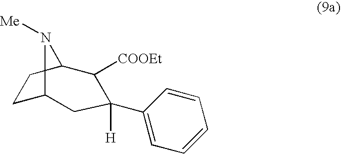

(2R,3S)-8-Methyl-3-phenyl-8-azabicyclo[3.2.1]octane-2-carboxylic acid ethyl ester (9a): A solution of 7a (1.5 g; 7.7 mmol) in anhydrous toluene was added to a stirred solution of phenylmagnesiumbromide (15.4 mmol) in anhydrous Et₂O (15 mL) at −40° C., such that the internal temperature was kept between −20° C. and −40° C. The reaction mixture was stirred at −20° C. to −40° C. for 90 min, or until TLC indicated complete transformation of starting material. The reaction mixture was poured onto a mixture of conc. HCl (2 mL) and ice (20 mL) and stirred for 20 min. The aqueous phase was washed with Et₂O (2×30 mL), and made alkaline to pH 10-11 using 4 M NaOH. The aqueous phase was extracted using CH₂Cl₂ (4×30 mL) and the combined organic fractions dried (MgSO₄), filtered and evaporated to dryness to yield 1.9 g 9a as an oil.

(2S,3R)-8-Methyl-3-phenyl-8-azabicyclo[3.2.1]octane-2-carboxylic acid ethyl ester (9b): Was synthesised similarly from 7b.

### Example 5

General Procedure for Obtaining Pure Trans-Isomer by Isomerisation

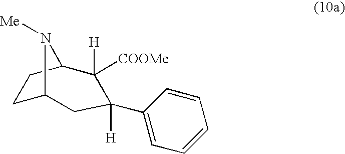

(2R,3S)-8-Methyl-3-phenyl-8-azabicyclo[3.2.1]octane-2-carboxylic acid ethyl ester (10a): A mixture of 9a (1.9 g; 7.0 mmol), NaOMe (780 mg; 2 mmol) and anhydrous MeOH (30 mL) was refluxed over night and then evaporated to dryness. Water (20 mL) was added, and extracted using CH₂Cl₂ (3×30 mL). The combined organic fractions were dried (MgSO₄), filtered and evaporated to dryness to yield 1.0 g 10a as an oil.

(2S,3R)-8-Methyl-3-phenyl-8-azabicyclo[3.2.1]octane-2-carboxylic acid ethyl ester (10b): Was synthesised similarly from 9b.

### Example 6

General Procedure for Conducting Step 3

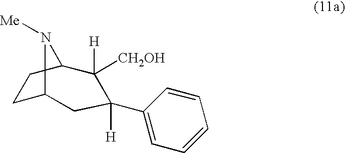

(2R,3S)-(8-Methyl-3-phenyl-8-aza-bicyclo[3.2.1]oct-2-yl)-methanol (11a): A suspension of LiAlH₄ (190 mg; 5.1 mmol) in anhydrous THF (10 mL) was slowly added to a solution of 10a (1.0 g; 3.9 mmol) in anhydrous THF (20 mL) cooled to −40° C. The reaction mixture was stirred at −40° C. for 2 h and then carefully added a solution of 2.5 mL H₂O and 2.5 mL 1 M NaOH. The alkaline mixture was stirred and then extracted using CH₂Cl₂ (4×30 mL) and the combined organic fractions dried (MgSO₄), filtered and evaporated to dryness to yield 860 mg 11a as an oil.

(2S,3R)-(8-Methyl-3-phenyl-8-aza-bicyclo[3.2.1]oct-2-yl)-methanol (11b): Was synthesised similarly from 10b.

### Example 7

General Procedure for Conducting Step 4

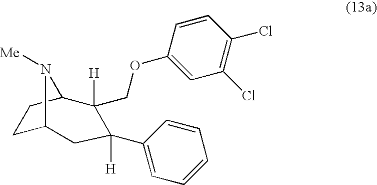

(2R,3S)-2-(3,4-Dichlorophenoxymethyl)-8-methyl-3-phenyl-8-aza-bicyclo[3.2.1]octane (13a): A solution of 11a (850 mg; 3.7 mmol), p-toluenesulfonylchloride (810 mg; 4.3 mmol) and triethylamine (590 mg; 4.3 mmol) in anhydrous CH₂Cl₂ (15 mL) was stirred for 2.5 h at rt. The reaction mixture was added H₂O (20 mL), the organic fraction isolated and the aqueous phase extracted using CH₂Cl₂ (2×30 mL). The combined organic fractions were washed with sat. NaHCO₃, dried (MgSO₄), filtered and evaporated to dryness to yield 1.2 g O-tosylated intermediate 12a.

A stirred solution of 3,4-dichlorophenol (780 mg; 4.8 mmol) in DMF (5 mL) was cooled to −10° C. and added NaH (116 mg 60% susp.; 4.8 mmol). The suspension was allowed to warm up to rt. and stirred for 30 min. A solution of 12a (1.2 g; 3.2 mmol) in DMF (10 mL) was added and the reaction mixture was heated to 100° C. for 3 h. The reaction mixture was cooled to rt, added 3 M CaCl₂ (aq.; 50 mL) and extracted using Et₂O (2×30 mL). The combined organic fractions were dried (MgSO₄), filtered and evaporated to dryness. Column chromatography (acetone:MeOH:NH₃ (1% aq.)=9:1:1) yielded 710 mg product. The isolated product was dissolved in Et₂O:MeOH (9:1; 10 mL) and added a solution of fumaric acid (27 mL; 0.0775 M) in Et₂O:MeOH (9:1). The solution was left at rt. over night and the product was isolated by filtration to yield 780 mg 13a.

M.p.: 211.4-212.4° C.

GCMS (free base, Cl, methane): 376 (M+1)

H-NMR [500 MHz, DMSO-d₆ (ref. 2.50)]: 7.45 (d, 1H); 7.30 (m, 4H); 7.20 (m, 1H); 7.0 (d, 1H); 6.75 (dd, 1H); 6.53 (s, 2H); 3.80 (m, 1H); 3.73 (m, 1H); 3.63 (m, 1H); 3.48 (m, 1H); 2.73 (m, 2H); 2.55 (s, 3H); 2.22-1.95 (m, 4H); 1.85 (m, 1H); 1.70 (m, 1H).

(2S,3R)-2-(3,4-Dichloro-phenoxymethyl)-8-methyl-3-phenyl-8-aza-bicyclo[3.2.1]octane (13b): Is synthesised similarly from 11b.

M.p., GCMS and H-NMR as for 13a.

### Example 8

By Use of the General Procedure,

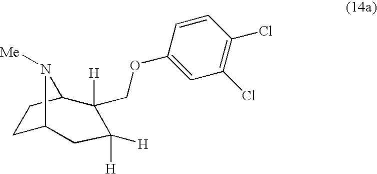

(2R)-2-(3,4-Dichloro-phenoxymethyl)-8-methyl-8-aza-bicyclo[3.2.1]octane (14a) was synthesised as the fumarates.

M.p.: 176-180° C.

GCMS (free base, Cl, methane): 300 (M+1).

H-NMR [500 MHz, DMSO-d₆ (ref. 2.50)]: 7.53 (d, 1H); 7.25 (d, 1H); 6.98 (d, 1H); 6.48 (s, 1H); 3.85 (m, 2H); 3.55 (m, 2H); 2.5 (s, 3H); 2.5 (m, 1H); 2.1 (m, 1H); 1.93 (m, 2H); 1.65 (s, 1H); 1.55 (m, 2H); 1.25 (m, 1H).

(1S,2S,5R)-2-(3,4-Dichloro-phenoxymethyl)-8-methyl-8-aza-bicyclo[3.2.1]octane fumaric acid salt:

Was prepared according to the general procedure from (+)-Ecgonine ethylester. M.p.: 176-180° C.

### Example 9

By Use of the General Procedure,

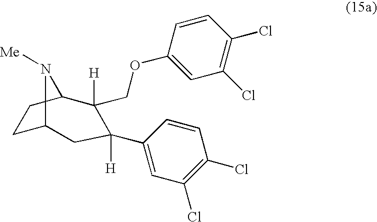

(2R,3S)-2-(3,4-Dichloro-phenoxymethyl)-3-(3,4-dichloro-phenyl)-8-methyl-8-aza-bicyclo[3.2.1]octane (15a) and (2S,3R)-2-(3,4-Dichloro-phenoxymethyl)-3-(3,4-dichloro-phenyl)-8-methyl-8-aza-bicyclo[3.2.1]octane (15b): was synthesised as the fumarates.

M.p.: 215.1-217.2° C.

GCMS (free base, Cl, methane): 444 (M+1).

H-NMR [500 MHz, DMSO-d₆ (ref. 2.50)]: 7.70 (d,₁H); 7.55 (d, 1H); 7.45 (d, 1H); 7.30 (dd, 1H); 7.00 (d, 1H); 6.75 (dd, 1H); 6.55 (s, 2H); 3.80 (m, 1H); 3.70 (m, 1H); 3.60 (m, 1H); 3.55 (m, 1H); 2.80 (m, 1H); 2.70 (m, 1H); 2.55 (s, 3H); 2.15 (m, 1H); 2.05 (m, 3H); 1.80 (m, 1H); 1.70 (m, 1H).

### Example 10

The following further compounds were synthesised using the general procedure.

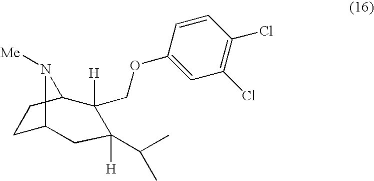

(2R,3S)-2-(3,4-Dichloro-phenoxymethyl)-3-(2-propyl)-8-methyl-8-aza-bicyclo[3.2.1]octane fumaric acid salt (16): Was synthesised, using the general procedure.

M.p.: 152.3-154.5° C.

(1S,2S,3R,5R)-2-(3,4-Dichloro-phenoxymethyl)-3-(2-propyl)-8-methyl-8-aza-bicyclo[3.2.1]octane fumaric acid salt: Was synthesised, using the general procedure, starting from (+)-ecgonine ethylester (7b).

M.p.: 148.5-150.5° C.

(2R,3S)-2-(3,4-Dichloro-phenoxymethyl)-3-(2-propyl)-8-H-8-aza-bicyclo[3.2.1]octane fumaric acid salt:

Prepared by the standard procedure for demethylation of (2R,3S)-2-(3,4-dichloro-phenoxymethyl)-3-(2-propyl)-8-methyl-8-aza-bicyclo[3.2.1]octane, using 1-chloroethyl-chloroformate.

M.p.: 185-186° C.

(2R,3S)-2-(3,4-Dichloro-phenoxymethyl)-3-(2-propyl)-8-(2-propyl)-8-aza-bicyclo[3.2.1]octane fumaric acid salt:

A mixture of (2R,3S)-2-(3,4-dichloro-phenoxymethyl)-3-(2-propyl)-8-H-8-aza-bicyclo[3.2.1]octane (0.4 g, 1.2 mmol), sodium hydride (55 mg, 1.3 mmol), 2-bromopropane (3.0 ml, 39.6 mmol) and tetrahydrofuran (15 ml) was stirred for 3 days at 55° C. The reaction mixture was cooled to rt, added 3 M CaCl₂ (aq.; 20 mL) and extracted using Et₂O (3×30 mL). The combined organic fractions were dried (MgSO₄), filtered and evaporated to dryness. Column chromatography (dichloromethane:MeOH:NH₃ (1% aq.)=9:1:1). The isolated product was dissolved in Et₂O:MeOH (9:1; 10 mL) and a saturated solution (1 eq.) of fumaric acid in Et₂O:MeOH (9:1) was added. The solution was left at rt. over night and the product was isolated by filtration to yield 230 mg (39%). M.p.: amorphous material.

(2R,3S)-2-(3,4-Dichloro-phenoxymethyl)-3-(1-butyl)-8-(2-propyl)-8-aza-bicyclo[3.2.1]octane fumaric acid salt:

Was prepared from (2R,3S)-2-(3,4-dichloro-phenoxymethyl)-3-(1-butyl)-8-H-8-aza-bicyclo[3.2.1]octane as described above. M.p.: 105-108° C.

(2R,3S)-2-(3,4-Dichloro-phenoxymethyl)-3-cycloheptyl-8-(2-propyl)-8-aza-bicyclo[3.2.1]octane fumaric acid salt:

Was prepared from (2R,3S)-2-(3,4-dichloro-phenoxymethyl)-3-cycloheptyl-8-H-8-aza-bicyclo[3.2.1]octane as described above. M.p.: amorphous material.

(2R,3S)-2-(3,4-Dichloro-phenoxymethyl)-3-(cycloheptyl)-8-methyl-8-aza-bicyclo[3.2.1]octane fumaric acid salt: Was synthesised, using the general procedure.

M.p.: 144-146° C.

(2R,3S)-2-(3,4-Dichloro-phenoxymethyl)-3-cycloheptyl-8-H-8-aza-bicyclo[3.2.1]octane fumaric acid salt:

Prepared by the standard procedure for demethylation of (2R,3S)-2-(3,4-dichloro-phenoxymethyl)-3-cycloheptyl-8-methyl-8-aza-bicyclo[3.2.1]octane, using 1-chloroethylchloroformate.

M.p.: 155-160° C.

(2R,3S)-2-(3,4-Dichloro-phenoxymethyl)-3-(1-butyl)-8-methyl-8-aza-bicyclo[3.2.1]octane fumaric acid salt: Was synthesised, using the general procedure.

M.p.: 123-125° C.

(2R,3S)-2-(3,4-Dichloro-phenoxymethyl)-3-(1-butyl)-8-H-8-aza-bicyclo[3.2.1]octane fumaric acid salt:

Prepared by the standard procedure for demethylation of (2R,3S)-2-(3,4-Dichloro-phenoxymethyl)-3-(1-butyl)-8-methyl-8-aza-bicyclo[3.2.1]octane, using 1-chloroethyl-chloroformate.

M.p.: 158-160° C.

(2R,3S)-2-(6-Chloropyridazin-3-yloxymethyl)-3-(2-propyl)-8-methyl-8-aza-bicyclo[3.2.1]octane fumaric acid salt: Was synthesised, using the general procedure directly from the alcohol precursor and 3,6-dichloropyridazine.

M.p.: 102.5-104.3° C.

(2R,3S)-2-(Pyrazin-2-yloxymethyl)-3-(2-propyl)-8-methyl-8-aza-bicyclo[3.2.1]octane fumaric acid salt: Was synthesised, using the general procedure directly from the alcohol precursor and 2-chloropyrazine.

M.p.: 148.8-151.1° C.

(2R,3S)-2-(6-Chlorobenzothiazol-2-yloxymethyl)-3-(2-propyl)-8-methyl-8-aza-bicyclo[3.2.1]octane fumaric acid salt: Was synthesised, using the general procedure directly from the alcohol precursor and 2,6-dichlorobenzothiazole.

M.p.: 168.5-169.8° C.

(2R,3S)-2-(6—Nitroquinolin-2-yloxymethyl)-3-(2-propyl)-8-methyl-8-aza-bicyclo[3.2.1]octane fumaric acid salt: Was synthesised, using the general procedure directly from the alcohol precursor and 2-chloro-6-nitro-quinoline. M.p.: 220-222° C.

(2R,3S)-2-(6-Chlorobenzothiazol-2-yloxymethyl)-3-(2-propyl)-8-H-8-aza-bicyclo[3.2.1]octane fumaric acid salt: Was synthesised, using the standard demethylation procedure on (2R,3S)-2-(6-chlorobenzothiazol-2-yloxymethyl)-3-(2-propyl)-8-methyl-8-aza-bicyclo[3.2.1]octane using diethylazadi- carboxylate as demethylation reagent. M.p.: 188.3-190.3° C.

(2R,3S)-2-(6-Chloro-pyrazin-2-yloxymethyl)-3-(2-propyl)-8-methyl-8-aza-bicyclo[3.2.1]octane fumaric acid salt: Was synthesised, using the general procedure directly from the alcohol precursor and 3-chloropyridazine. M.p.: amorphous material.

### Example 11

In Vitro Inhibition Activity

A number of compounds were tested for their ability to inhibit the reuptake of the monoamine neurotransmitters dopamine(DA) noradrenaline(NA) and serotonine(5-HT) in synaptosomes as described in WO 97/16451.

The test values are given as IC₅₀ (the concentration (μM) of the test substance which inhibits the specific binding of ³H-DA, ³H-NA, or ³H-5-HT by 50%).

Test results obtained by testing selected compounds of the present invention appear from the below table:

TABLE 1

| | DA- | NA- | 5-HT- |
|---|---|---|---|
| | uptake | uptake | uptake |
| Test compound | IC₅₀(μM) | IC₅₀(μM) | IC₅₀(μM) |
| Compound 15a; | | | |
| (2R,3S)-2-(3,4-Dichloro-phenoxy- | 0.015 | 0.032 | 0.020 |
| methyl)-3-(3,4-dichloro-phenyl)-8- | | | |
| methyl-8-aza-bicyclo[3.2.1]octane | | | |
| fumaric acid salt | | | |
| Compound 13a; | | | |
| (2R,3S)-2-(3,4-Dichlorophenoxy- | 0.54 | 0.80 | 0.0004 |
| methyl)-8-methyl-3-phenyl-8-aza- | | | |
| bicyclo[3.2.1]octane (13a | | | |
| Compound 16; | | | |
| (2R,3S)-2-(3,4-Dichloro-phenoxy- | >1.0 | 2.7 | 0.00072 |
| methyl)-3-(2-propyl)-8-methyl-8-aza- | | | |
| bicyclo[3.2.1]octane fumaric acid salt | | | |
| Compound of Example 10 | | | |
| (2R,3S)-2-(3,4-Dichloro-phenoxy- | 0.26 | 0.15 | 0.009 |
| methyl)-3-(2-propyl)-8-H-8-aza- | | | |
| bicyclo[3.2.1]octane fumaric acid salt | | | |

1. A compound of general formula (I), 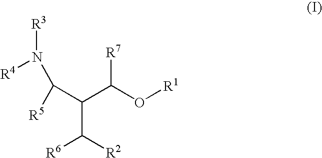
or a pharmaceutically acceptable addition salt thereof or the N-oxide thereof wherein
R¹ is aryl or heteroaryl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R² is hydrogen, alkyl, cycloalkyl, alkenyl, alkynyl, —O-alkyl, —S-alkyl, —NR⁸-alkyl, aryl or heteroaryl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl; and
with the proviso that when R² is hydrogen, optionally substituted aryl or optionally substituted heteroaryl, then R¹ is 3,4-dichlorophenyl;
R⁸ is hydrogen, alkyl, alkenyl, or alkynyl; and
R³ is hydrogen, alkyl, alkenyl, alkynyl, cycloalkyl, cycloalkylalkyl, 2-hydroxyethyl, aryl, arylalkyl, heteroaryl or heteroarylalkyl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R⁴ is hydrogen, alkyl, alkenyl, alkynyl, cycloalkyl, cycloalkylalkyl, 2-hydroxyethyl, aryl, arylalkyl, heteroaryl or heteroarylalkyl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R⁵ is hydrogen;
R⁶ is hydrogen;
R⁷ is hydrogen; or
one of R³ and R⁴ form together with R⁶—(CH₂)ₚ—;
wherein p is 2 or 3;
the other of R³ and R⁴ is hydrogen, alkyl, alkenyl, alkynyl, cycloalkyl, cycloalkylalkyl, 2-hydroxyethyl, aryl, arylalkyl, heteroaryl or heteroarylalkyl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R⁵ is hydrogen;
R⁷ is hydrogen; or
one of R³ and R⁴ form together with R⁶—(CH₂)_{q}—CR⁹—(CH₂)ᵣ—;
wherein q is 0 or 1;
r is 0 or 1;
the other of R³ and R⁴ is hydrogen, alkyl, alkenyl, alkynyl, cycloalkyl, cycloalkylalkyl, 2-hydroxyethyl, aryl, arylalkyl, heteroaryl or heteroarylalkyl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R⁹ together with R⁵ form —(CH₂)ₛ—;
wherein s is 2 or 3;
R⁷ is hydrogen; or
one of R³ and R⁴ form together with R⁶—(CH₂)_{q}—CR⁹—(CH₂)ᵣ—;
wherein q is 0 or 1;
r is 0 or 1;
R⁹ together with R⁵ form —(CH₂)ₛ—;
wherein s is 1, 2, or 3;
the other of R³ and R⁴ form together with R⁷—(CH₂)ₜ—;
wherein t is 1, 2, 3, or 4.

2. The compound according to claim 1, wherein
R³ is hydrogen, alkyl, alkenyl, alkynyl, cycloalkyl, cycloalkylalkyl, 2-hydroxyethyl, aryl, arylalkyl, heteroaryl or heteroarylalkyl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R⁴ is hydrogen, alkyl, alkenyl, alkynyl, cycloalkyl, cycloalkylalkyl, 2-hydroxyethyl aryl, arylalkyl, hetroaryl or heteroarylalkyl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R⁵ is hydrogen;
R⁶ is hydrogen;
R⁷ is hydrogen;
and R¹, R², and R⁸ are defined as in claim 1.

3. The compound according to claim 1, wherein
one of R³ and R⁴ form together with R⁶—(CH₂)ₚ—;
wherein p is 2 or 3;
the other of R³ and R⁴ is hydrogen, alkyl, alkenyl, alkynyl, cycloalkyl, cycloalkylalkyl, 2-hydroxyethyl, aryl, arylalkyl, heteroaryl or heteroarylalkyl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R⁵ is hydrogen;
R⁷ is hydrogen;
and R¹, R², and R⁸ are defined as in claim 1.

4. The compound according to claim 3 of general formula (II) 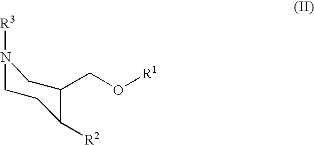
wherein R¹, R², and R³ are defined as in claim 1.

5. The compound according to claim 1, wherein
one of R³ and R⁴ form together with R⁶—(CH₂)_{q}—CR⁹—(CH₂)ᵣ—;
wherein q is 0 or 1;
r is 0 or 1;
the other of R³ and R⁴ is hydrogen, alkyl, alkenyl, alkynyl, cycloalkyl, cycloalkylalkyl, 2-hydroxyethyl, aryl, arylalkyl, heteroaryl or heteroarylalkyl;
wherein the aryl and heteroaryl are optionally substituted with one or more substituents selected from the group consisting of:
halogen, hydroxy, amino, cyano, nitro, trifluoromethyl, alkoxy, cycloalkoxy, alkyl, cycloalkyl, cycloalkylalkyl, alkenyl, alkynyl, aryl and heteroaryl;
R⁹ together with R⁵ form —(CH₂)ₛ—;
wherein s is 2 or 3;
R⁷ is hydrogen;
and R¹, R², and R⁸ are defined as in claim 1.

6. The compound according to claim 5 of general formula (III) 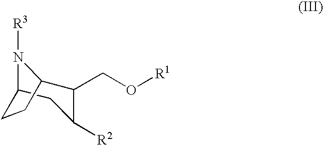
wherein R¹, R², and R³ are defined as above.

7. The compound according to claim 6, wherein
R¹ is aryl or heteroaryl; wherein the aryl and heteroaryl are optionally substituted with one or more substituents selected from the group consisting of:
halogen, hydroxy, amino, cyano, nitro, trifluoromethyl and alkyl;
R² is hydrogen, alkyl, cycloalkyl, or aryl; wherein the aryl is optionally substituted with one or more substituents selected from the group consisting of:
halogen, hydroxy, amino, cyano, nitro, trifluoromethyl, and alkyl;
with the proviso that when R² is hydrogen or optionally substituted aryl, then R¹ is 3,4-dichlorophenyl; and
R³ is hydrogen or alkyl.

8. The compound according to claim 1, wherein
one of R³ and R⁴ form together with R⁶—(CH₂)_{q}—CR⁹—(CH₂)ᵣ—;
wherein q is 0 or 1;
r is 0 or 1;
R⁹ together with R⁵ form —(CH₂)ₛ—;
wherein s is 1, 2, or 3;
the other of R³ and R⁴ form together with R⁷—(CH₂)ₜ—;
wherein t is 1, 2, 3, or 4;
and R¹, R², and R⁸ are defined as in claim 1.

9. The compound according to claim 8 of general formula (IV) 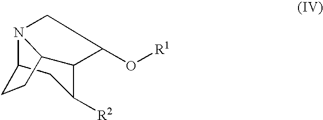
wherein R¹ and R² are defined as in claim 1.

10. A compound of claim 1 which is
(2R,3S)-2-(3,4-Dichlorophenoxymethyl)-8-methyl-3-phenyl-8-aza-bicyclo[3.2.1]octane;
(2S,3R)-2-(3,4-Dichloro-phenoxymethyl)-8-methyl-3-phenyl-8-aza-bicyclo[3.2.1]octane;
(2R)-2-(3,4-Dichloro-phenoxymethyl)-8-methyl-8-aza-bicyclo[3.2.1]octane;
(1S,2S,5R)-2-(3,4-Dichloro-phenoxymethyl)-8-methyl-8-aza-bicyclo[3.2.1]octane;
(2R,3S)-2-(3,4-Dichloro-phenoxymethyl)-3-(3,4-dichloro-phenyl)-8-methyl-8-aza-bicyclo[3.2.1]octane;
(2S,3R)-2-(3,4-Dichloro-phenoxymethyl)-3-(3,4-dichloro-phenyl)-8-methyl-8-aza-bicyclo[3.2.1]octane;
(2R,3S)-2-(3,4-Dichloro-phenoxymethyl)-3-(2-propyl)-8-methyl-8-aza-bicyclo[3.2.1]octane;
(1S,2S,3R,5R)-2-(3,4-Dichloro-phenoxymethyl)-3-(2-propyl)-8-methyl-8-aza-bicyclo[3.2.1]octane;
(2R,3S)-2-(3,4-Dichloro-phenoxymethyl)-3-(2-propyl)-8-H-8-aza-bicyclo[3.2.1]octane;
(2R,3S)-2-(3,4-Dichloro-phenoxymethyl)-3-(2-propyl)-8-(2-propyl)-8-aza-bicyclo[3.2.1]octane;
(2R,3S)-2-(3,4-Dichloro-phenoxymethyl)-3-(1-butyl8-(2-propyl)8-aza-bicyclo[3.2.1]octane;
(2R,3S)-2-(3,4-Dichloro-phenoxymethyl)-3-cycloheptyl-8-(2-propyl)-8-aza-bicyclo[3.2.1]octane;
(2R,3S)-2-(3,4-Dichloro-phenoxymethyl)-3-(cycloheptyl8-methyl-8-aza-bicyclo[3.2.1]octane;
(2R,3S)-2-(3,4-Dichloro-phenoxymethyl)-3-cycloheptyl-8-H-8-aza-bicyclo[3.2.1]octane;
(2R,3S)-2-(3,4-Dichloro-phenoxymethyl)-3-(1-butyl)-8-methyl-8-aza-bicyclo[3.2.1]octane;
(2R,3S)-2-(3,4-Dichloro-phenoxymethyl)-3-(1-butyl)-8-H-8-aza-bicyclo[3.2.1]octane;
(2R,3S)-2-(6-Chloropyridazin-3-yloxymethyl)-3-(2-propyl)-8-methyl-8-aza-bicyclo[3.2.1]octane;
(2R,3S)-2-(Pyrazin-2-yloxymethyl)-3-(2-propyl)-8-methyl-8-aza-bicyclo[3.2.1]octane;
(2R,3S)-2-(6-Chlorobenzothiazol-2-yloxymethyl)-3-(2-propyl)-8-methyl-8-aza-bicyclo[3.2.1]octane;
(2R,3S)-2-(6-Nitroquinolin-2-yloxymethyl)-3-(2-propyl)-8-methyl-8-aza-bicyclo[3.2.1]octane;
(2R,3S)-2-(6-Chlorobenzothiazol-2-yloxymethyl)-3-(2-propyl)-8-H-8-aza-bicyclo[3.2.1]octane;
(2R,3S)-2-(6-Chloro-pyrazin-2-yloxymethyl)-3-(2-propyl)-8-methyl-8-aza-bicyclo[3.2.1]octane;
or a pharmaceutically acceptable addition salt thereof.

11. A pharmaceutical composition, comprising a therapeutically effective amount of a compound of any one of claims 1-10, or a pharmaceutically acceptable addition salt thereof, together with at least one pharmaceutically acceptable carrier, excipient or diluent.

12. The use of a compound according to any one of claims 1-10, or a pharmaceutically acceptable addition salt thereof, for the manufacture of a pharmaceutical composition for the treatment, prevention or alleviation of a disease or a disorder or a condition of a mammal, including a human, which disease, disorder or condition is responsive to inhibition of monoamine neurotransmitter re-uptake in the central nervous system.

13. The use according to claim 12, wherein the disease, disorder or condition is depression, pseudodementia, Ganser's syndrome, obsessive compulsive disorders, panic disorders, memory deficits, attention deficit hyperactivity disorder, obesity, anxiety or eating disorders.

14. A method for treatment, prevention or alleviation of a disease or a disorder or a condition of a living animal body, including a human, which disorder, disease or condition is responsive to inhibition of monoamine neurotransmitter re-uptake in the central nervous system, which method comprises the step of administering to such a living animal body in need thereof a therapeutically effective amount of a compound according to any one of the claims 1-10.

15. The method of claim 14, wherein the disease, disorder or condition is depression, pseudodementia, Ganser's syndrome, obsessive compulsive disorders, panic disorders, memory deficits, attention deficit hyperactivity disorder, obesity, anxiety or eating disorders.
